(19) United States
(12) Patent Application Publication
(10) Pub. No.: US 2002/0193851 A1
(43) Pub. Date: Dec. 19, 2002

(54) Energy treatment apparatus for treating gastrointestinal tract and method for using same

(76) Inventors: David E. Silverman, Palo Alto, CA (US); Alan Stein, Moss Beach, CA (US)

Correspondence Address: FLEHR HOHBACH TEST ALBRITTON & HERBERT LLP, Four Embarcadero Center, Suite 3400, San Francisco, CA, 94111-4187, US

(21) Appl. No.: 09/881,144

(22) Filed: Jun. 14, 2001

(51) Int. Cl. A61B 18/18

(52) U.S. Cl. 607/101; 606/41

(57) ABSTRACT

An apparatus for treating a portion of a wall forming a cavity in a body having a natural body opening for accessing the cavity. The apparatus includes an elongate probe member, a suction source, and an energy source. The elongate probe member includes proximal and distal extremities. The distal extremity of the elongate probe member has an outer surface, at least one recess opening onto the outer surface, and an internal passageway communicating with the recess. The suction source is coupled to the proximal extremity of the elongate probe member and is in communication with the passageway to create suction in the recess for drawing the portion of the wall into the recess. At least one stylet is carried by the elongate probe member and is extendable into the recess. The energy source is coupled to the stylet for delivering energy such as radio frequency energy through the stylet to the portion of the wall for treatment thereof.

### BACKGROUND OF THE INVENTION

1. Field of the Invention

This invention pertains to the treatment of a portion of a wall forming a cavity in a body. More particularly, this invention pertains to an apparatus and a method for delivering energy to a portion of the wall forming the gastrointestinal tract for treatment thereof.

2. Description of Related Art

Gastroesophageal reflux disease (GERD) is a failure of the anti-reflux barrier, allowing abnormal reflux of gastric contents into the esophagus of the gastrointestinal tract. Gastroesophageal reflux disease is a disorder which is usually characterized by a defective lower esophageal sphincter (LES), a gastric emptying disorder occurring with or without failed esophageal peristalsis. The disease usually manifests itself during “transient lower esophageal sphincter relaxation” episodes, the frequency of which is greatly increased in patients who reflux.

Minimally invasive procedures have been provided for treating the wall of the gastrointestinal tract to treat GERD and other ailments. For example, U.S. Pat. No. 6,056,744 to Edwards discloses a sphincter treatment apparatus including an energy delivery device introduction member having a plurality of arms and an expansion device. U.S. Pat. No. 6,073,052 to Zelickson et al. discloses a device and method for treatment of gastroesophageal reflux disease. The device disclosed by the Zelickson patent includes an insertion device, an energy source, and an energy transmitting device.

### Objects and Summary of the Invention

In general, it is an object of the present invention to provide a minimally invasive apparatus and method for delivering energy to a portion of a wall forming a cavity in a body, such as the gastrointestinal tract, for treatment thereof.

Another object of the invention is to provide an apparatus and method of the above character for delivering radio frequency energy to the portion of such wall.

Another object of the invention is to provide an apparatus and method of the above character for providing repeatable treatments to the portion of such wall.

Another object of the invention is to provide an apparatus and method of the above character in which a recess in the apparatus is utilized to shape the portion of such wall into a protrusion into which the energy is delivered.

Another object of the invention is to provide and apparatus and method of the above character in which suction is utilized to draw the portion of such wall into the recess in the apparatus.

Another object of the present invention is to provide an apparatus and method of the above character in which the apparatus guides a stylet into the protrusion.

Another object of the present invention is to provide an apparatus and method of the above character which can be used for treating gastroesophageal reflux disease (GERD).

In general, an apparatus in accordance with the present invention is disclosed for treating a portion of a wall forming a cavity in a body having a natural body opening for accessing the cavity. The apparatus includes an elongate probe member, a suction source, and an energy source. The elongate probe member includes proximal and distal extremities. The distal extremity of the elongate probe member has an outer surface, at least one recess opening onto the outer surface, and an internal passageway communicating with the recess. The suction source is coupled to the proximal extremity of the elongate probe member and is in communication with the passageway to create suction in the recess for drawing the portion of the wall into the recess. At least one stylet is carried by the elongate probe member and is extendable into the recess. The energy source is coupled to the stylet for delivering energy such as radio frequency energy through the stylet into the portion of the wall for treatment thereof.

### BRIEF DESCRIPTION OF THE DRAWINGS

FIG. 1 is a perspective view of an apparatus for treating a portion of a wall forming a cavity in a body in accordance with the present invention.

FIG. 2 is a cross-sectional view of the apparatus of FIG. 1 taken along line 2-2 of FIG. 1.

FIG. 3 is a cross-sectional view of a portion of the apparatus of FIG. 1 taken along line 3-3 of FIG. 1.

FIG. 4 is an enlarged view of a distal portion of the needle assembly of the apparatus of FIG. 1.

FIG. 5 is an elevational view of the apparatus of FIG. 1 treating a lower esophageal sphincter in accordance with the present invention.

FIG. 6 is a cross-sectional view of the lower esophageal sphincter of FIG. 5 at the level of the gastric cardia taken along the line 6-6 of FIG. 5.

FIG. 7 is a perspective view of another embodiment of a distal portion of the apparatus of the present invention.

FIG. 8 is a perspective view of yet another embodiment of a distal portion of the apparatus of the present invention.

FIG. 9 is a perspective view of a further embodiment of a distal portion of the apparatus of the present invention.

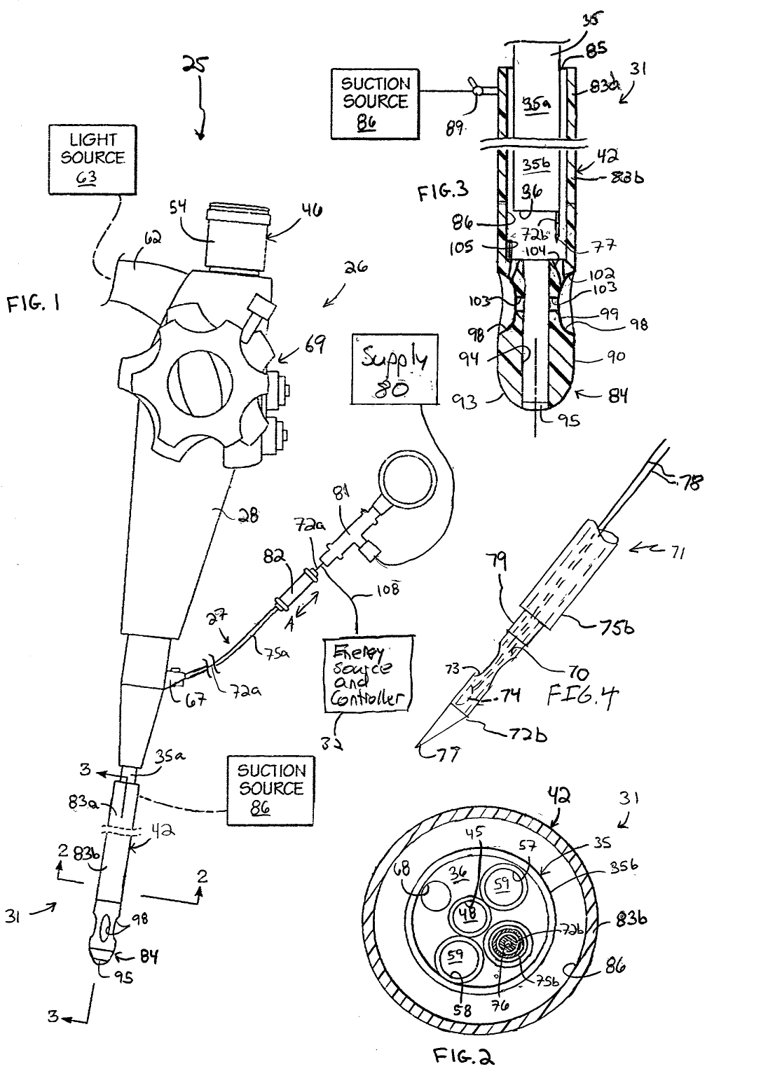

FIG. 1O is a perspective view of another embodiment of a distal portion of the apparatus of the present invention.

### BRIEF DESCRIPTION OF THE PREFERRED EMBODIMENTS

Reference will now be made in detail to the preferred embodiments of the invention, examples of which are illustrated in the accompanying drawings. While the invention will be described in conjunction with the preferred embodiments, it will be understood that they are not intended to limit the invention to those embodiments. On the contrary, the invention is intended to cover alternatives, modifications and equivalents, which may be included within the spirit and scope of the invention as defined by the appended claims.

In general, a medical or treatment apparatus 25 is provided for treating a portion of a wall forming a cavity in a body which has a natural body opening for accessing the cavity. Treatment apparatus 25 generally includes a probe device 26 and an optional stylet assembly 27. Apparatus 25 has a handle means or handle 28, and an elongate probe member or probe member 31 extending from the handle. Stylet assembly 27 is carried by probe device 26 and is operably connected to a suitable energy source and controller 32.

In one embodiment shown in FIG. 1, a conventional or other suitable gastroscope or endoscope can be used for probe 26. The exemplary medical probe or scope 26 shown in FIGS. 1-3 is an Olympus CF Type 40 L/I endoscope made by Olympus Corporation of Tokyo Japan. Medical probe or scope 26 includes a flexible elongate tubular member in the form an insertion tube 35 having a proximal extremity or end 35a, a distal extremity or end 35b, and a distal face 36. A handle means or handle assembly is coupled to proximal extremity 35a of elongate insertion tube 35 and includes probe handle 28. The insertion tube 35 includes a plurality of bores or passageways extending axially from proximal extremity 35a to distal extremity 35b. A plurality of five such passageways, including a central passageway 37, are shown in FIG. 2.

In the embodiment shown in FIG. 1, treatment apparatus 25 includes an overtube assembly 42 used in combination with medical probe 26 and stylet assembly 27 to deliver energy to a portion of the wall for treatment thereof. Overtube assembly 42 is included in the elongate probe member 31 of apparatus 25 and is removably mounted on the distal extremity of probe device 26 and preferably on distal extremity 35b of the insertion tube.

An optical viewing device 46 is formed integral with probe device 26 and has an optical element or objective lens 48 carried by the central passageway 37 of insertion tube 35, as shown in FIG. 2. Lens 48 has a field of view at distal face 36 which permits the operator to view forwardly of insertion tube distal extremity 35b. Optical viewing device 36 further includes an eye piece 54 mounted on a proximal end of probe handle 37. Second and third illumination passageways 57 and 58 are provided within insertion tube 35 peripherally of central passageway 37 for carrying respective light fiber assemblies or light guides 59. A connection cable 62, a portion of which is shown in FIG. 1, extends from probe handle 37 to a conventional light source 63. First and second light guides 59 extend through insertion tube 35 and cable 62 for providing illumination forwardly of distal face 36.

A working passageway or channel 66 is further provided in insertion tube 35 and extends to a side port 67 formed in probe handle 37. An additional passageway 68 extends through insertion tube 35 and can be used as an air and/or water inlet or outlet or a lumen for providing suction. Insertion tube 35 is flexible so as to facilitate its insertion and advancement through a body. Distal extremity 35b is bendable for selectively directing distal face 36 in a desired direction. A plurality of finger operable controls 69 are provided on probe handle 37 for, among other things, operating the bendable distal extremity 35b of insertion tube 35 and the supply and removal of fluids through the insertion tube 35.

Stylet assembly 27 shown in FIGS. 1-3 is a needle assembly and can be of any conventional type such as a sclerotherapy needle similar to the Bard ® Flexitip™ needle manufactured by C.R. Bard, Inc. of Billerica, Md., as modified for energy delivery. Stylet assembly 27 includes a stylet 71 having a needle member or needle 72 provided with a proximal end portion 72a and a distal end portion 72b. Needle 72 can be made from metal such as stainless steel and/or any other material suitable for conducting energy. Stylet 71 may include an optional sleeve member or sleeve 75 having a proximal end portion or extremity 75a and a distal end portion or extremity 75b. Sleeve or elongate tubular member 75 is made from any suitable material such as flexible plastic or metal and has a lumen extending longitudinally therethrough for receiving needle 72. Sleeve 75 and needle 72 are slidable relative to each other in a longitudinal direction. In this regard, tubular needle 72 is slidably disposed in sleeve 75 and movable from a retracted position in which distal needle end portion 72b is recessed within distal end portion 75b to an extended position in which needle 72 projects distally of sleeve 75. Needle 72 and sleeve 75 can be slidably disposed within working channel 66 and side port 67 of insertion tube 35 and each have a length so that when distal end portions 72b and 75b are extending from distal extremity 35b of insertion tube 35 or otherwise in the vicinity of distal face 36, proximal end portions 72a and 75a are accessible adjacent side port 67.

Needle 72 is optionally provided with a lumen or internal passage 76 extending longitudinally there through for carrying liquids or other materials through the hollow or tubular needle (see FIG. 4). Internal passage 76 extends longitudinally through the needle from proximal end portion 72a to distal end portion 72b. Needle distal end portion 72b is made from any suitable material such as stainless steel and has a size ranging from 16 to 28 gauge and preferably ranging from 21 to 26 gauge. Preferably, the distal end portion 72b has a sharpened or beveled distal end 77 formed in part by a tapered end surface. At least one opening 73 is provided in distal end portion 72b and can include an opening on beveled distal end 77. As an alternative to or in addition to the opening on beveled distal end 77, the needle can include at least one or more openings 73 in its cylindrical wall.

An optional temperature sensor or sensors, such as thermocouple 74, can be disposed in internal passageway 76, and secured therein by any suitable means such as an adhesive, in close proximity to one or more openings 73. Electrical leads 78 extend from the temperature sensor 74 to the proximal extremity of stylet assembly 27 for electrically coupling the sensor to a controller or other suitable processor and/or monitor. The optional temperature sensor or sensors can be carried by the stylet assembly 27 in or along the needle 72 and/or in or along the sleeve 75.

An optional coating or layer 79 of an insulating material can be formed on the distal extremity of needle 72, as shown in FIG. 4. Coating 79 is disposed proximal of the one or more openings 73 and preferably extends proximally along a sufficient length of the needle so as to extend into sleeve 75 during both retraction and full operable extension of the distal end portion 72b of the needle relative to sleeve 75. It should be appreciated that other needle configurations may also be used. For example, the needle may be provided with a sharpened or pointed distal end which is generally conical in shape and has no opening.

Optionally, the apparatus of the present invention may also be configured to inject an implant-forming material into the portion of the wall to form an implant therein. When so configured, treatment apparatus 25 further includes a supply 80 of the implant-forming material, which, depending on the type of implant-forming material used, can include one or more syringes or other containers. Where the implant-forming material requires the injection of a fluid, a fluid connector 81 is secured or coupled to proximal end portion 72a of the needle and fluidly connects the supply 80 to needle 72. Fluid connector 81 includes suitable fitting portions which communicate with passage 76 within needle 72.

A gripping member 82 is secured to the proximal end portion 75a of sleeve 75. Fluid connector 81 and gripping member 82 are longitudinally movable relative to each other, as indicated by arrow A in FIG. 1, so as to cause relative longitudinal movement between needle 72 and sleeve 75. More specifically, gripping member 82 can be slid forwardly and rearwardly on proximal end portion 72a of needle 72 relative to fluid connector 81. Movement of gripping member 82 forwardly relative to fluid connector 81 causes distal end portion 75b of sleeve 75 to extend fully over distal end portion 72b of needle 72 so that the needle has fully retracted within sleeve 75. Conversely, movement of gripping member 82 rearwardly relative to fluid connector 81 causes sleeve distal end portion 75b to retract relative to needle distal end portion 72b so as to expose needle distal end portion 72b.

Exemplary implant-forming materials include any suitable material from which an implant can be formed when a fluid, separately or in conjunction with another fluid, is introduced into the tissue of an body. Suitable implant-forming materials suitable for use with the apparatus of the present invention are described in U.S. Pat. Nos. 5,667,767 dated Sep. 16, 1997, 5,580,568 dated Dec. 3,1996, and 5,695,480 dated Dec. 9, 1997, and in International Publication Number WO 97/45131 having an International Publication Date of Dec. 4, 1997, the entire content of each of which is incorporated herein by this reference. Other suitable implant-forming materials include suitable suspensions such as injectable bioglass of the type described in Walker et al., “Injectable Bioglass as a Potential Substitute for Injectable Polytetrafluorethylene Particles” , J. Urol., 148:645-7, 1992, small particle species such as polytetrafluoroethylene (PTFE) particles in glycerine such as POLYTEF®, biocompatible compositions comprising discrete, polymeric and silicone rubber bodies such as described in U.S. Pat. Nos. 5,007,940, 5,158,573 and 5,116,387 to Berg and biocompatible compositions comprising carbon coated beads such as disclosed in U.S. Pat. No. 5,451,406 to Lawin. Such suitable materials for forming implants further include collagen and other biodegradable material of the type disclosed in U.S. Pat. No. 4,803,075 to Wallace et al. and other known injectable materials.

Overtube assembly 42 of the present invention includes an elongate tubular overtube member 83 including a proximal end portion 83a, a distal end portion 83b, and an end cap 84 as shown in FIGS. 1-3. Overtube member 83 may be formed of either rigid or flexible materials. For example, overtube member 83 may be formed of rigid plastic tubing, flexible plastic tubing or flexible silicon tubing and can be made from any suitable material such as polyetheretherketone (PEEK), polypropylene (PP) or fluorinated ethylene propylene (FEP). Preferably, overtube member 83 is optically clear and is flexible such that it provides a tight frictional seal against insertion tube 35 at proximal portion 83a and/or at distal portion 83b adjacent end cap 84. Overtube assembly 42 has a length preferably approximate the length of insertion tube 35, as for example a length ranging from 75 to 250 centimeters and preferably approximately 150 centimeters, and a diameter ranging from 0.5 to 5.0 centimeters and preferably ranging from 0.75 to 1.5 centimeters. One should appreciate, however, the length of overtube assembly 42 may vary depending upon the size of the probe device, the intended patient and other factors.

The overtube assembly 42 is rotatably mounted on at least a portion of insertion tube 35. An seal 85 having an aperture is provided on overtube member 83 and is shown as being provided on the proximal end of the overtube member 83. Insertion tube 35 may be inserted into overtube assembly 42 through the aperture of seal 85 thereby forming an internal chamber or pressure chamber 86 between insertion tube 35 and overtube assembly 42. A suction source 87 is coupled with overtube assembly 42 via a suction source coupling 89 to produce negative pressure within pressure chamber 86. Suction source 87 may include any well known suction device such as a suction pump or a conventional 50 cc syringe.

End cap 84 is secured to distal end portion 83b of overtube member 83 by any suitable means such as heat sealing, adhesive, threads or press fit. End cap 84 has a length ranging from 1 to 10 cm and preferably ranging from 2 to 3 cm and may be formed by injection molding or machining from any suitable material. The end cap 84 is preferably made from a clear plastic such as polymethylpentene (PMP) or acrylic so as to be transparent. The end cap 84 has an outer surface preferably in the form of outer cylindrical surface 90 and an outer diameter approximately equal to the outer diameter of overtube member 83. End cap 84 includes a rounded end or blunt nose 93 which facilitates insertion into and advancement through the gastrointestinal tract thus preventing or minimizing injury to thereto.

Optical lens 48 of probe device 26 has a field of view that includes a portion of a pressure chamber 86 which is formed within a distal end 83b of overtube member 83 and the portion of an end cap 84 that are located forwardly of distal extremity 35b of the insertion tube. End cap 84 is provided with a central passageway or bore 94 that communicates with chamber 86 and terminates at an opening formed in blunt nose 93. An optical window 95 made from any suitable material is secured to blunt nose 93 at the opening to enable optical viewing device 36 to enhance visual feedback of the gastrointestinal tract beyond end cap 84.

End cap 84 is formed with at least one recess or vacuum cavity 98 that opens onto outer surface 90. One should appreciate that one, two, three, four or more recesses or vacuum cavities 98 may be provided in the distal extremity of overtube assembly 42. Such vacuum cavities can be circumferentially disposed about end cap 84, as shown in FIG. 1 where three of four circumferentially spaced-apart cavities 98 are shown and as shown in FIG. 6 where four circumferentially spaced-apart cavities 98 are shown. Each of the vacuum cavities 98 of end cap 84 is elongated and is formed in part by a recessed wall or cavity base 99 of a predetermined depth. A peripheral wall 102 serves as the side wall of vacuum cavity 98.

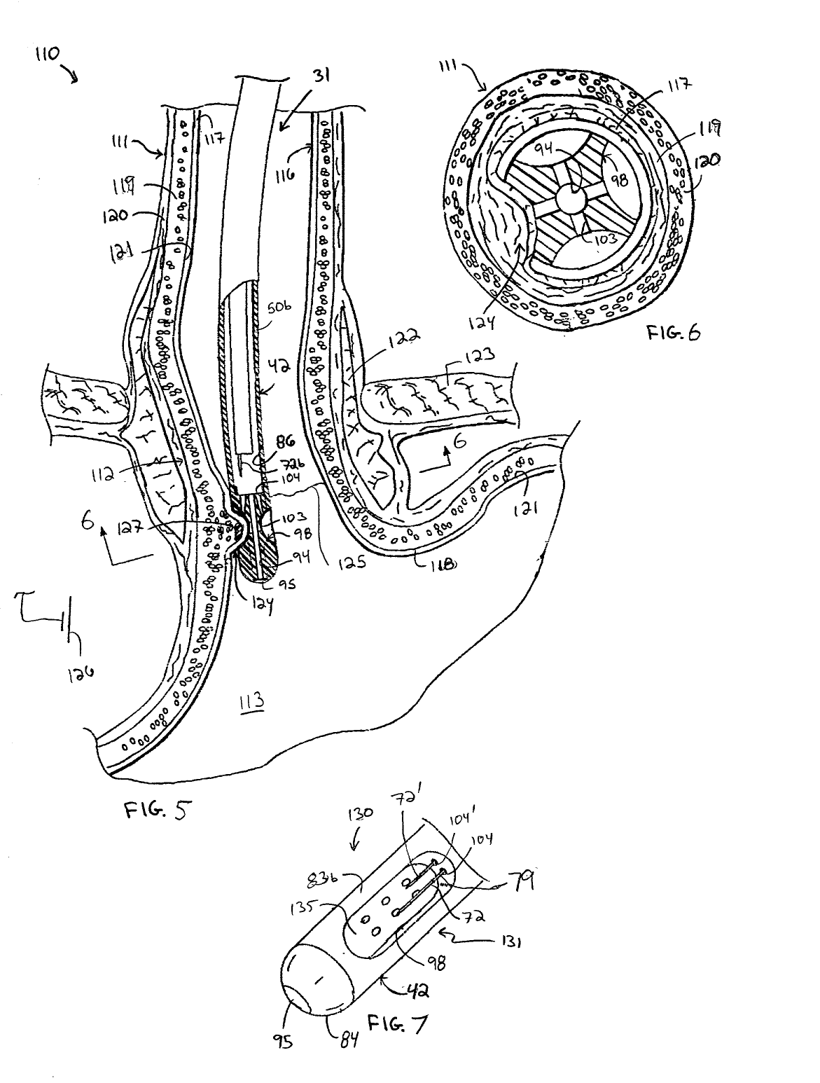

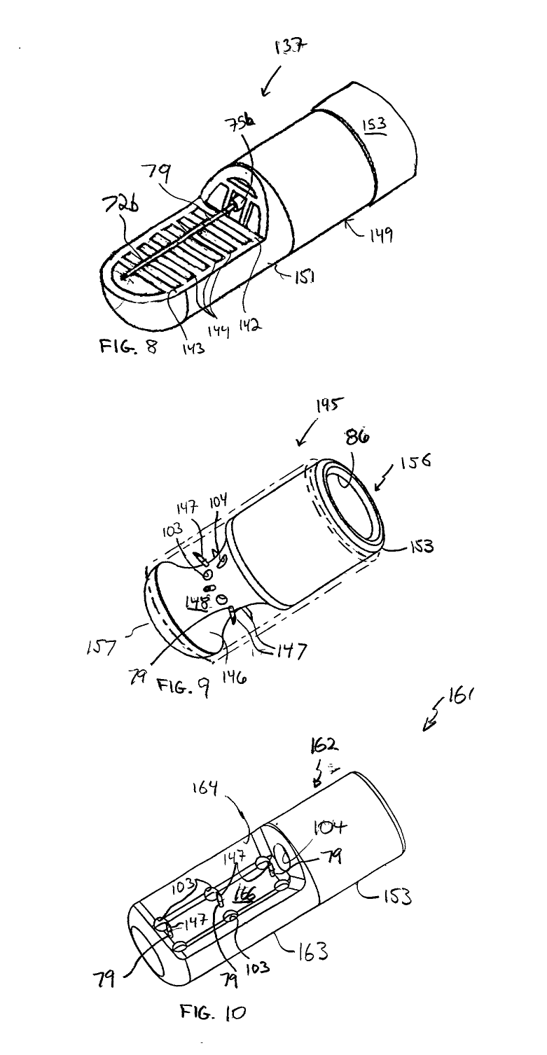

One should appreciate that the size and shape of the one or more vacuum cavities may vary in accordance with the medical procedure with which it is used. For example, vacuum cavity may have a semispherical shape as shown in FIG. 3. Each vacuum cavity 98 is fluidly connected to pressure chamber 86 by means of at least one passageway 103 which extends from central bore 94 and forms a vacuum opening located in cavity base 99, as shown in FIG. 3. One should appreciate that a one or more passageways 103 can be provided for each cavity base 99. For example, a plurality of passageways 103 can be provided for each cavity base 99. Although cavity base 99 is shown as being concave, it should be appreciated that the cavity base can take the form of various other shapes, for example planar and convex, and fall within the scope of the present invention.

Insertion tube 35 extends through overtube member 83 such that distal extremity 35b is adjacent to distal end 83b(see FIGS. 1 and 3). Because optical lens 48 of probe device 26 has a field of view that includes a portion of pressure chamber 86 formed within distal end 83b of overtube member 83 and the portion of the transparent end cap 84 that is located forwardly of insertion tube distal extremity 35b, including optical window 95, optical viewing device 36 provides visual feedback about needle 72 when the needle is extended from distal face 36 of insertion tube 35 into vacuum chamber 86 and vacuum cavity 98.

Overtube assembly 42 may include a needle guide 104 for each of the four vacuum cavities 98. The embodiment shown in FIG. 3 includes four corresponding needle guides 104. An identifying reference mark such as a spline 105 may be provided on the inside of overtube member 83 adjacent distal end 83b that is viewable through optical viewing device 46 to define a reference point for determining the position of needle 72 with respect to the four vacuum cavities. Use of identifying mark 105 provides a relative position, for example a twelve o'clock position, of insertion tube 35 with respect to overtube assembly 42. Any other suitable identifying reference mark, such as an etched and/or inked mark on the inside of overtube member 83, can alternatively be provided.

The stylet assembly 27 of treatment apparatus 25 is operably connected to energy source and controller 32. As shown schematically in FIG. 1, energy source 32 is operably connected to needle 72 by a suitable energy source transmission conductor 108, one example being a cable. Transmission conductor 108 is connected to proximal end portion 72a and can include electrical leads 78 to optional thermocouple 74. Thus, needle 72 serves as an energy transmitting conduit that extends through port 67 and down passageway 66 of insertion tube 35 and into vacuum cavity 98.

Energy source and controller 32 preferably includes a radio frequency (RF) source or generator coupled to needle 72 which acts as a radio frequency active electrode. Energy source 32 generates and transmits energy at a level sufficient to heat targeted tissue to a temperature of at least 50° C., preferably in the range of 65° C. and 80° C., within a predetermined time period ranging from one second to ten minutes and preferably ranging from 45 to 90 seconds. In particular, radio frequency energy flowing through targeted tissue causes heating of the tissue due to absorption of the radio frequency energy delivered from electrode or antenna 72 by the targeted tissue and ohmic heating due to electrical resistance of the targeted tissue. The energy source and controller 32 also has the ability to monitor the impedance between the one or more active electrodes, such as needle 72, and the indifferent or other return electrode of apparatus 25.

In operation and use, treatment apparatus 25 can be used for any suitable procedure within the upper gastrointestinal tract, such as the treatment of gastroesophageal reflux disease (GERD). A portion of a human body 110 is shown in FIGS. 5 and 6 and has an internal cavity in the form of esophagus 111 extending through a lower esophageal sphincter 112 to a stomach 113. Such cavity is accessible by a natural body opening in the form of a mouth and is defined by an intraluminal wall 116. Esophagus 111 is part of the gastrointestinal tract of body 110 that extends from the mouth to an anus. An esophageal mucosa 117 serves as the inner layer of intraluminal wall 116 in the esophagus 111 and gastric mucosa 118 serves as the inner layer of the intramural wall 116 in stomach 113. The esophageal mucosa and the gastric mucosa meet at a squamocolumnar junction 125.

Wall 116 has a muscle layer comprising a layer of circular muscle 119 extending beneath mucosa layers 117 and 118 and a layer of longitudinal muscle 120 extending beneath circular muscle 119. The muscle layers 119 and 120 each extend around esophagus 111 and stomach 113. Wall 116 further includes a submucosal layer or submucosa 121 extending between the mucosa and the muscle layers. A submucosal space, that is a potential space, can be created between mucosa layer 117 or 118 and circular muscle layer 119 by the separation of the respective mucosa layer from muscle layer 120. In addition, as with any muscle, wall 116 includes an intramuscular potential space, that is a space which can be created intramuscularly by distension and separation of muscle fibers within a single muscle or between layers of muscle. Wall 116 has a depth or thickness which includes at least mucosa layers 117 and 118, muscle layers 119 and 120 and submucosa 121. A phreno-esophageal ligament 122 and a diaphragm 123 extend around esophagus 111 above lower esophageal sphincter 112.

Probe device 26 is prepared by connecting light cable 62 to light source 63 and attaching the proper eyepiece 54 to probe handle 37. In addition, all other conventional attachments are applied to probe device 26. Insertion tube 35 is then inserted within overtube assembly 42 via the aperture of seal 85.

After the patient has been appropriately sedated or anesthetized, probe handle 37 is grasped by the physician to introduce distal end 83b of overtube assembly 42 and distal extremity 35b of insertion tube 35 into the mouth of the patient and to advance overtube assembly 42 with insertion tube 35 down esophagus 111. Optical viewing device 36 facilitates advancement by the physician of the insertion tube 35 and overtube assembly 42. In addition, the optical viewing device 36 enables the physician to ensure that overtube assembly 42 is properly disposed within esophagus 111. Insertion tube 35 and overtube assembly 42 each preferably have a length so that when distal extremity 35b and distal end 83b are in the vicinity of lower esophageal sphincter 112, proximal extremity 35a and proximal end 8a are outside of body 110. The optically clear material of end cap 84 permits light from light guides 59 to illuminate the esophagus and thus enhance visualization by optical viewing device 36 through window 95.

Although the method of positioning overtube assembly 42 within the esophagus is described herein as utilizing an optical viewing device, it should be appreciated that the overtube assembly can be introduced into the esophagus without the aid of an optical viewing device. For example, the overtube assembly can be positioned in the esophagus by merely introducing the distal end of the overtube assembly a predetermined distance to the desired treatment site. The insertion distance of overtube assembly can be measured by external observation of the proximal extremity of assembly and optionally by gradations provided on the outer surface of such proximal extremity.

A portion of the procedure for treating wall 116 in the vicinity of lower esophageal sphincter 112 is shown in FIG. 5. Under the guidance of optical viewing device 36, which has a field of view forward distal face 36 of insertion tube 35 and forward of overtube assembly 42 through optical window 95, overtube assembly 42 is maneuvered to a position such that at least one of the vacuum cavities 98 is adjacent the portion of wall 116 which is to be treated, that is a portion of wall 116 adjacent lower esophageal sphincter 112. Suction source 87 is then activated to draw air from and evacuate pressure chamber 86 of overtube assembly 42. A negative pressure is thus created within the pressure chamber 86 and the vacuum cavities 98. This negative pressure creates a suction effect which draws targeted tissue, that is a portion of wall 116 to be treated, into vacuum cavity 98 to shape the targeted tissue into a protrusion 124, as shown in FIG. 5. It should be appreciated that FIGS. 5 and 6 are somewhat schematic and that, in this regard, the size of esophagus 111 has been exaggerated relative to the size of insertion tube 35 and overtube 42 in FIG. 5. The sizing of esophagus 111 relative to insertion tube 35 and overtube 42 are more accurate in FIG. 6, where the insertion tube 35 is shown as having a diameter that approximates the diameter of the esophagus 111.

Distal end portions 72b and 75b are now advanced until such distal end portions of stylet 71 are in the vicinity of insertion tube distal extremity 35b, overtube distal end 83b and end cap 84. Needle or antenna 72 and sleeve 75 are each movable from a first position in which distal end portions 72b and 75b are each retracted within end cap 84 and thus not extending into vacuum cavity 98 to a second position in which the distal end portions 72b and 75b extend into the vacuum cavity 98. The needle and sleeve each have a sufficient length so that the physician can extend both the needle and the sleeve distally from distal extremity 35b and into the desired vacuum cavity. The physician retracts sleeve 75 relative to needle 72 by means of an adjustment mechanism so that needle distal end portion 72b extends beyond sleeve distal end portion 75b a selected amount of at least one millimeter and preferably ranging from 2 to 15 millimeters.

The physician advances sleeve 75 and needle 72 distally from insertion tube distal extremity 35b into a respective needle guide 104 such that sleeve 75 and needle 72 are proximate to the protrusion 124. The physician extends needle 72 and optional coating 79 through needle guide 104 into vacuum cavity 98, which is occupied by the portion of wall 116 to be treated, by moving the needle 72 and sleeve 75 closer to side port 67. This causes the sharpened end of needle 72 and optional coating 79 to penetrate protrusion 124. The field of view of optical viewing device 36 permits the physician to observe movement of needle 72 into needle opening 104 and, in some cases, penetration of protrusion 124. The optically clear material of end cap 84 permits light guides 59 to illuminate the field of view and thus enhance visualization through the end cap.

The predetermined depth of cavity base 99 and the shape of the vacuum cavity 98 determine the layer of wall 166 into which needle 72 is introduced. As shown in FIG. 5, cavity base 99 has an appropriate depth below the outer cylindrical surface of end cap 84 such that needle 72 extends through mucosal layer 117 and into the submucosal layer 121 of the wall 116. The protective insulating coating 79 extends through the mucosa 117. It is noted that the amount of extension of needle 72 into vacuum cavity 98 can be determined by correlating the relative movement between proximal extremities of the needle and sleeve which can be calibrated in a known manner. As can be seen, the layer of wall 116 into which needle 72 is introduced and the depth of such penetration can easily be predetermined. The predetermined depth of cavity base 99 and the extent of penetration of needle 72 can be adjusted to accommodate the desired treatment.

After distal end 72b of the needle has penetrated protrusion 124, the physician activates energy source 32. Energy source 32 generates and transmits radio frequency energy to the exposed energy transmitting distal end 72b of needle 72 which serves as an active electrode and transmits radio frequency energy into a portion of wall 116 of the gastrointestinal tract, namely the portion of protrusion 124 penetrated by and immediately adjacent distal end 72b and not protected by insulating coating 79. An indifferent electrode patch or ground pad electrode 126, schematically shown in FIG. 5, is applied to the body and electrically connected to energy source 32. Ground pad electrode 126 serves as a return electrode and forms the other electrical contact and complete an electrical circuit. Optional thermocouple 74 permits the temperature of the tissue being treated to be monitored during the application of radio frequency or other energy. Such temperature can be used by controller 32 for adjusting or terminating the amount of energy being supplied to needle 72 by any number of conventional control algorithms. As discussed above, the controller 32 can also monitor the impedance between needle 72 and ground electrode 126 for conventionally controlling the supply of energy to the needle and thus the lesion being formed in the treated tissue.

The targeted tissue of protrusion 124 absorbs the radiated radio frequency energy which, in turn, generates heat within the targeted tissue. As the esophageal wall, and particularly the submucosal layer 121, is composed in part of collagen, the heating of collagen tissue within an appropriate temperature range results in a tightening or shrinkage of the collagen tissue. In this manner, wall 116 is tightened in the vicinity of lower esophageal sphincter 112. It is also desirable to deliver sufficient energy such that lesions within protrusion 124 are produced having a sufficient magnitude and area to cause an infiltration of lesion by fibroblasts, myofibroblasts, macrophages and other cells involved in the tissue healing process. In effect, these cells cause a contraction of tissue within protrusion 124 which alters the biomechanical properties of lower esophageal sphincter 112 so as to result in a tightening thereof. Such lesions formed in wall 116 may also act in a nonmechanical manner to alter the characteristics of the wall by modifying sensory motor nervous feedback and control in the wall. It should be appreciated that such a modification of sensory motor nervous feedback and control can be accomplished by an application of energy to such portion of the body wall alone or in conjunction with the formation of implants in the body wall.

When preferably protected by optional coating 79, the mucosa 117 is shielded from the energy introduced into wall 116 by needle electrode 72. Accordingly, the opening in the mucosa 117 created by needle 72 is minimized and more easily healed. In an alternate method of the invention, sleeve 75 is introduced into wall 116 and, separately or together with optional coating 79, serves to protect the intervening tissue between the exposed portion of the needle 72 and the gastrointestinal tract, for example mucosa 117, to protect such intervening tissue from the energy of needle electrode 72 in the same manner as discussed above. When sleeve 75 is used from protecting mucosa 117, the sleeve is preferably formed with a tapered distal end 75b for facilitating the sleeve's entry into wall 116.

As noted above, the treatment apparatus 25 can be used to form one or more implants 127 in wall 116 for further treating the gastrointestinal tract. Optionally, a saline solution or any other aqueous solution may be injected into wall 116 before or after implant formation. For example, such solution may be injected into wall 116 before implant formation to cause a local edema and further enlarge protrusion 124 which, in turn, assists penetration of the needle into the protrusion.

An implant-forming material can be introduced into the wall 116 of the gastrointestinal tract to form at least one implant 127 therein. In one method for forming such an implant, overtube assembly 42 and treatment apparatus 25 are used to form a protrusion in a portion of wall 116 in manner similar to that discussed above and an implant-forming material is injected into protrusion 124. In fact, the implant-forming material can be injected into the same protrusion to which energy was applied. One should appreciate that the introduction of the implant-forming material into wall 116 can be monitored transabdominally or transesophageally by ultrasound.

Although only a single implant 127 in wall 116 in the vicinity of the lower esophageal sphincter 112 is shown in FIG. 5, additional implants may be created in wall 116. In preparation thereof, needle 72 is removed from protrusion 124, vacuum cavity 98 and needle guide 104. The physician then rotates insertion tube 35 with respect to overtube assembly 42 to align needle 72 with another desired needle guide 104 in order to penetrate a different protrusion. Insertion tube distal extremity 35b is then positioned within overtube assembly 42 such that needle 72 aligns with another needle guide 104 corresponding to the other cavity 98 and the procedure discussed above is repeated thus treating one or more portions of wall 116. The physician may rotate insertion tube 35 with respect to overtube assembly 42 to align needle 72 with the desired needle guide 104. For example, the physician may align needle 72 with the needle guide 104 that is shown on the right in FIG. 5 when the corresponding vacuum cavity 98 is positioned against the portion of wall 116 on the right side of esophagus 111.

One should appreciate that a multitude of implant configurations are possible. For example, a plurality of solid implants may be disposed in a plane which extends substantially perpendicularly to a longitudinal axis extending along the centerline of esophagus 111. Alternatively, the plurality of solid implants may be disposed in a nonplanar configuration and disclosed above and/or below and/or at the lower esophageal sphincter 112. It should be appreciated that one or more implants can be formed in portions of the wall other than the mucosal layers. For example, one or more implants can be formed in one or both of or between the muscle layers 119 and 120. Such implants can serve to augment or partially or completely coapt the esophagus in the vicinity of the lower esophageal sphincter and can also serve to reduce the distensibility of the muscle layers. Implants formed within or between muscle layers 119 and 120 can be arranged in a variety of configurations, including any of the various configuration of implants described above.

Although the method of the invention has been described as including the formation of a space by a saline solution injected into wall 116 prior to an injection of implant-forming material into wall 116, it should be appreciated that the space can be formed by other aqueous or physiologic solutions or by a local anesthetic. It is also noted that injection of an aqueous or other solution prior to injection of the implant-forming material is not essential. It is within the scope of the present invention, for example, to inject the implant-forming material directly into protrusion 124 without the prior formation of a the space by an injection of saline solution or otherwise. The implant-forming material can also be injected directly into wall 116 without an injection of saline or any other solution for any secondary purpose described herein or otherwise. A saline or other aqueous or physiologic solution can optionally be introduced into a space formed by the implant-forming material, that is after the introduction of the implant-forming material into wall 116, to facilitate formation of the implant and acceptance of the implant by the body. It can thus be seen that the invention is broad enough to cover the introduction of any conditioning solution, for example such an aqueous or physiologic solution, into the tissue before, during or after the treatment to facilitate the treatment.

In an alternative method for forming a plurality of implants within wall 116, a plurality of spaces can be formed by saline solution from syringe 80. Subsequently, the implant-forming material from syringe 80 can be sequentially injected into each of such spaces. It should also be appreciated that the implants of the present invention can be used as delivery vehicles for other materials such as medicaments, for example

radioisotopes, chemotherapeutic agents, anti-inflammatory agents and/or antibiotics.

The apparatus described above can be used in other gastrointestinal procedures for other than the treatment of gastroesophageal reflux disease and be within the scope of the present invention. For example, similar apparatus can be used in the vicinity of other muscles in a body such as muscles in the vicinity of the anal sphincter to treat incompetent anal sphincters as disclosed in copending U.S. patent application Ser. No. 09/286,245, filed Apr. 5, 1999.

The present invention encompasses a minimally invasive apparatus and method for delivering radio frequency energy to a portion of a wall forming a cavity in a body to be treated. The present invention promotes a minimally invasive treatment having consistent and repeatable results. The apparatus and method of the present invention is particularly well suited for the treatment of gastroesophageal reflux disease (GERD).

A recess in the apparatus shapes the portion of such wall into a protrusion into which the energy is delivered. The apparatus may additionally be utilized to form an implant in the same protrusion, however, one should appreciate that an implant need not be formed in the same protrusion. In fact, the apparatus and method of the present invention may be utilized to deliver energy to one protrusion at a first location and to form an implant in another protrusion at a second location. Alternatively, the apparatus and method of the present invention may be used to treat a portion of a wall by only delivering energy or by only forming one or more implants The structure of the probe member may vary and be within the scope of the present invention. For example, a plurality of needles may be provided in either the insertion tube and/or the overtube assembly. In another embodiment of the treatment apparatus of the present invention, a modified treatment apparatus 130 having an end cap 131 is provided. End cap 131 includes two needles 72 and 72′ for each vacuum cavity 98 as shown in FIG. 7. Like reference numerals have been used to describe like components of end caps 84 and 131. Needles of differing lengths and/or colors may be used in order to distinguish one needle from the other as a physician views the distal ends of the needles through optical viewing device 36, and an optional coating 79 can be provided on one or both of needles 72 and 72′. The treatment apparatus 130 shown in FIG. 7 also includes a vacuum cavity 98 located in distal end 83b of the overtube member which has a flat cavity base 135 instead of concave cavity base 99 but is otherwise similar to the overtube assembly shown in FIGS. 1-3.

In operation and use, treatment apparatus 130 may operated in either a bipolar or a monopolar mode. Each of needle electrodes 72 and 72′ shown in FIG. 7 can be operably connected to energy source 32. In the monopolar mode of operation, electrodes 72 and 72′ serves as active electrodes and are used in combination with an indifferent electrode patch or ground pad electrode 126 (not shown in FIG. 7) that is applied to the body to form the other electrical contact and complete an electrical circuit. In the bipolar mode of operation, electrode 72 is an active electrode and electrode 72′ is a return electrode whereby a ground pad is not necessary. Active electrode 72 is used in combination with return electrode 72′ to complete the electrical circuit.

One should appreciate that energy source 32 may have multiple channels, delivering separately modulated power to each active electrode. This reduces nonuniform heating that occurs when more energy is delivered through one of the stylers to a zone of greater conductivity and less heating occurs around other stylers which are placed into less conductive tissue. In the event that both needles 72 and 72′ are utilized as active electrodes, the active electrodes can be multiplexed in order to treat an entire protrusion drawn into the vacuum cavity or only a portion thereof, for example, active electrodes 72 and 72′ can deliver radio frequency energy to protrusion 124 simultaneously or sequentially. One should appreciate that if the targeted tissue is uniform, a single channel radio frequency energy source may be used to provide energy to the multiple stylers for generating lesions which are relatively uniform in size.

A portion of a modified probe member 137, similar to probe member 31 above, is shown in FIG. 8. Like reference numerals have been used to describe like components of probe members 31 and 137. Probe member 31 includes a short tubular assembly or overtip assembly 149, that is used in combination with a conventional probe instead of the overtube assembly that is shown in FIGS. 1-3. More specifically, overtip assembly 149 is removably attached or mounted to distal end 35b of insertion tube 35 (not shown in FIG. 8). As such, overtip assembly 149 does not extend along the length of insertion tube 35 and, in use with the insertion tube, does not have a proximal portion accessible outside of the human body. In this regard, overtip assembly 149 has a length ranging from one to ten centimeters and preferably ranging from two to three centimeters. Overtip assembly 149 includes a cylindrical body or end cap 151 made from plastic or any other suitable material. Overtip assembly has a diameter ranging from 0.5 to 5.0 centimeters and preferably ranging from 0.75 to 1.5 centimeters.

Modified probe member 137 includes a single needle 72 and a layer of insulating material, such as sleeve 75, slidably disposed on the needle 72. An optional coating 79 can be provided on the distal extremity of the needle 72 for protecting intervening tissue in the body wall being treated. End cap 151 has a recess 142 which does not have a peripheral side wall similar to side wall 102 shown in FIG. 3. Instead, recess 142 is formed by a flat recess base 143. A plurality of vacuum passageways 144 open onto flat recess base 143 which are fluidly connected to the pressure chamber 86 (not shown in FIG. 8) of probe member 137.

A flexible tubular member or sleeve 153, as shown in FIG. 8, or other suitable means is included for removably mounting the end cap 151 of overtip assembly 149 to distal end 35b of insertion tube 35. Sleeve 153 is made from any suitable material such as silicone, and is diametrically sized and has sufficient elasticity to extend over a portion of distal extremity 35b of the insertion tube 35 and secure thereto with a friction fit. The proximal end of overtip assembly 149 generally abuts distal end 35b when the overtip assembly is so secured to the insertion tube. Sleeve 153 further serves as a seal and thus serves to provide a fluid-tight connection between insertion tube 35 and end cap 151 of overtip assembly 149. At least one longitudinally-extending needle guide 104 is provided in the proximal end of end cap 151 and extends to the proximal end of vacuum cavity or recess 142 for permitting needle 72 and sleeve 75 to be removably inserted into the vacuum cavity or recess of overtip assembly 149. Needle 72 and sleeve 75 are thus movable relative to end cap 151 from a first position, in which the needle and sleeve do not extend into recess 142, and a second position shown in FIG. 8, in which the needle and sleeve both extend into the recess 142.

In operation and use, overtip assembly 149 is mounted on distal end 35b of the insertion tube 35 prior to insertion of the tube 35 into the body 110. Suction is provided to internal chamber 83 and thus to vacuum cavity or recess by means of insertion tube 35 when it is desired to draw a portion of wall 116 into vacuum cavity or recess. When suction source 86 is activated to draw air from and evacuate pressure chamber 86, a negative pressure is created within the pressure chamber 86 and vacuum passageways 144. This negative pressure creates a suction effect which draws targeted tissue, that is a portion of wall 116 to be treated, into recess 142 and against flat recess base 143 to shape the targeted tissue complementary to recess 143. The physician may then penetrate the targeted tissue with stylet 138 and treat wall 116 in a similar manner as discussed above with respect to apparatus 25. Needle 72, slidably carried by insertion tube 35 and manipulable from outside of body 110, is inserted into the protrusion 124 for delivering energy to protrusion 124 and/or injecting the implant-forming material into protrusion 124 in the manner discussed above. Coating 79 and/or sleeve 75 can be used for protecting the intervening tissue of wall 166, such as mucosa 117, from the radio frequency or other energy introduced into the wall 116 by exposed portion of needle 72.

In an alternate embodiment, an apparatus similar to apparatus 25 is provided in which one or more suitable stylers, each of which can be similar to stylet 71 disclosed above, are slidably carried by overtube assembly 42. For example, one or more longitudinally-extending bores can be provided in the tubular wall of overtube assembly 42 for carrying such one or more stylers. Each such bore communicates with an opening in one or more of the vacuum cavities 98 so that each stylet can be extended into a vacuum cavity 98 and retracted therefrom.

In other embodiments of the treatment apparatus of the present invention circumferential recess geometries may also be used to shape the protrusion in various annular shapes for treatment thereof. A modified probe member 145 having such a circumferential recess is shown in FIG. 9. Like reference numerals have been used in FIG. 9 to describe like components of probe members 31, 137 and 145. Probe member 145 includes an overtip assembly 156 having an end cap 157. A circumferential vacuum recess 146 formed with a base 148 is provided in the end cap 157.

Overtip assembly 156 includes a plurality of stylers 147 that can extend radially from the base 148 of recess 146. Each of the stylers 147 is substantially similar to stylet 71 and includes a needle 72 and a sleeve 75 concentrically disposed around the proximal portion of the needle. An optional coating 79 can be provided on the distal extremity of the needle 72. Suitable materials for the stylers 147 include, but are not limited to, stainless steel, other stainless steels, and other suitable materials known to those skilled in the art. The stylers 147 are slidably carried by insertion tube 25 and accessible from the proximal extremity of the probe member 137. The stylers are electrically coupled with energy source 32 by any suitable means including a transmission cable. End cap 157 is formed with a plurality of vacuum passageways 103, one for each stylet 147, and one or more needle guides 104 for guiding the stylers into the end cap 157. Vacuum passageways 103 each have a curved portion, not shown, for directing the respective stylet 147 radially from base 148 into the circumferential vacuum cavity or recess 146.

Probe member 145 is used in substantially the same manner as probe member 137 discussed above. Vacuum recess 146 permits the formation of a circumferential protrusion from the wall of the gastrointestinal tract. One or more of the stylers 147 are then extended from base 148, either sequentially or in unison, into the protrusion. Suitable energy, such as radio frequency energy, is supplied to the one or more stylers 147 to treat the tissue exposed to the needle 72 in the manner discussed above. All stylers 147 may serve as active electrodes in a monopolar mode in which the device is used in combination with an indifferent electrode patch that is applied to the body to form the other electrical contact and complete an electrical circuit. In the event that the embodiment is utilized in a bipolar mode, at least two stylers are preferred. For example, every other stylet 147 can be configured as an active electrode and the remaining stylers 147 configured as return electrodes to complete the electrical circuit. Needle coating 79 and/or sleeve distal extremity 75b preferably extends through the inner layers of the gastrointestinal wall 116 for protecting such layers during treatment.

As discussed above, modified probe member 145 can be used in either monopolar or bipolar modes. Nonetheless, and although a plurality of stylers 147 are shown in FIG. 9, one should appreciate that any number of stylers may be provided. For example, in the case that the embodiment is utilized in a monopolar mode, only one stylet need be used.

Probe member 145 advantageously permits the formation of a circumferential lesion in the gastrointestinal or other intraluminal wall being treated. Such lesion can be formed in a single application of energy to the plurality of stylers, without need for rotating overtip assembly 156 within the lumen being treated.

One should appreciate that the circumferential vacuum recess can be provided with any suitable profile in order to form a circumferential protrusion of various desired shapes. Although the illustrated circumferential vacuum recess 146 extends completely around probe member 145, an arcuate vacuum recess can be provided that extends angularly about the longitudinal axis of the probe member less than the entire circumference of probe member 145. For example, vacuum recesses can be provided that extend approximately 90° or 180° around the probe member and be within the scope of the present invention.

Other embodiments of the present having an overtip assembly similar to overtip assembly 156 can be provided. For example, another embodiment of the probe member, not shown, has an overtube assembly, similar to overtube assembly 42, with a circumferential recess similar to recess 146. In such embodiment, the one or more stylers are slidably carried within the tubular wall of the overtube assembly and radially deflected by vacuum passageways 103 into the circumferential recess of the probe member.

In a further embodiment, overtip assembly 156 is modified so that the one or more stylers 147 are fixedly attached to base 148 of the circumferential recess 146. In such embodiment, an optional shield 158 is preferably provided for covering the extended stylers 147 during insertion of the overtip assembly 156 into the esophagus or other lumen of the body. Shield 158, shown in phantom lines in FIG. 9, is slidably disposed on end cap 157 and movable from a first or extended position, in which the shield extends over stylers 147 and recess 146 as shown in FIG. 9, and a second or retracted position, in which the shield is moved proximally on the end cap so as to expose the stylers 147 and recess 146. A suitable control mechanism (not shown) accessible from the proximal extremity of probe member 145 is included for moving the shield from its first position to its second position. In an exemplary embodiment, the control mechanism includes a pull wire extending from the shield 158 to the proximal end of probe member 145 for retracting the shield and a return spring for urging the shield back to its extended position. In operation, the radially-extending, fixed stylers 147 penetrate the protrusion formed within circumferential recess 146 as the tissue of the intraluminal wall is drawn into the recess 146.

In the embodiment of FIG. 10, a portion of a probe member 161 is depicted and includes an overtip assembly 162 having an end cap 163. The cylindrical end cap 163 has a geometry similar to the end cap 84 of probe member 31 shown in FIGS. 1-3 and other features similar to end cap 157 of probe member 145 shown in FIG. 9. Like reference numerals have been used to describe like components of probe members 31, 145 and 161. A longitudinally-extending vacuum cavity 164 is provided in end cap 163 and is formed in part by a base 166. A plurality of stylers 147 are extendable in a radial direction from the base 166. Although two rows of three stylers are shown in FIG. 10, one should appreciate that one or more stylers may be provided in various configurations, as is discussed with regard to probe member 137 shown in FIG. 9 above.

In one embodiment, the extendable stylers 147, which are accessible from the proximal end of probe member 161, are each movable from a first or retracted position in which the stylers do not extend into recess or cavity 164 and a second or extended position in which the stylers protrude radially from base 166 and thus extend into the cavity 164. Stylers 147 are shown in their second position in FIG. 10. Such movable stylers are slidably carried by insertion tube 35 of the probe member 26, but can also be slidably carried in the tubular wall of an overtube assembly, such as overtube assembly 42, when end cap 163 is part of such an overtube assembly. In another embodiment, stylers 147 are fixedly attached to base 166 and thus nonmovable relative to end cap 157. A longitudinally-movable shield, such as shield 158 discussed above with respect to probe member 145 is preferably provided in such an embodiment of probe member 161.

Probe member 161 is used in substantially the same manner as probe member 145 discussed above. An elongate protrusion is formed when the tissue to be treated is drawn into vacuum cavity 164. When movable stylers 147 are provided, the stylers are thereafter extended from base 166 into the protrusion and energy supplied to the stylers to treat the tissue of the protrusion, and thus the wall of the lumen in which end cap 157 is disposed. As discussed above, radio frequency energy is preferably supplied to stylers 147. An optional coating 79 is preferably provided on the distal extremities of the needles of the stylers 147 and, separately or together with the insulating sleeve of the stylers, serve to protect the intervening tissue in protrusion when radio frequency or other energy is provided to the stylers disposed in the protrusion. When a supply 78 of an implant-forming material is fluidly coupled to probe member 161, one or more implants can be formed in such wall in the manner discussed above.

The plurality of longitudinally-aligned stylers 147 of probe member 161 advantageously permits a longitudinal lesion in the gastrointestinal or other intraluminal wall being treated. Such lesion can be formed in a single application of energy to the plurality of stylers 147, without need for moving end cap 157 longitudinally or angularly within the esophagus or other body lumen being treated.

In the illustrated embodiments, the energy source utilized is a radio frequency source and the needle serves as a radio frequency electrode which is also referred to as an electrode 72 herein. Energy source 32, however, may employ various forms of energy generating devices other than a radio frequency source or generator. Such other sources include a source of light coupled to an optical fiber, a heated fluid coupled to a catheter with a channel configured to receive the heated fluid, a resistive heating source, a microwave source coupled to a microwave antenna, or an ultrasound power source coupled to an ultrasound emitter. Temperature and other feedback controls discussed above can be used with such other energy sources.

The treatment apparatus of the present invention can also be used for any suitable procedure within the lower gastrointestinal tract or any other lumen in a mammalian body. When used for treating an ailment of the lower gastrointestinal tract, such as fecal incontinence, a shorter probe member is utilized. Any of the end caps or overtubes disclosed herein, and equivalents thereof, each as appropriately sized for length and diameter, can be used for forming lesions in the wall of the lower gastrointestinal tract and for optionally forming implants in such wall.

Any of the apparatus disclosed herein can be modified to provide cooling of the intervening tissue in the wall of the gastrointestinal tract in which the protrusion is being formed so as to offset any heating of such intervening tissue from the energy being supplied to the needle electrode. Such cooling can supplement or be used in lieu of protecting such intervening tissue with optional coating 79 and/or the sleeve of the one or more stylers provided in such apparatus. Such optional cooling is particularly useful for protecting the mucosal layer of the internal body wall being treated. Any suitable liquid, such as water or saline, can be used for such cooling, and a supply of the cooling liquid is coupled to the proximal extremity of the apparatus.

The cooling liquid is preferably supplied to the mucosal layer by means of the stylet assembly, such as stylet assembly 27, of the apparatus. One or more longitudinally-extending lumens terminating in respective distal openings can be provided in the sleeve of the stylet assembly for carrying the coolant to the mucosal layer at the treatment site. The coolant is then sprayed from such openings on to the tissue in the vicinity of the needle penetration site. The cooling liquid can also be supplied through the central passageway of the sleeve. It should be appreciated that other means can be provided for supplying a cooling liquid with the apparatus to the exterior of the protrusion.

As can be seen from the foregoing, a minimally invasive apparatus and method provides a minimally invasive apparatus and method for delivering energy to a portion of a wall forming a cavity in a body, such as the gastrointestinal tract, for treatment thereof. The apparatus and method is preferably utilized in the upper portion of the gastrointestinal tract, for example in treating gastroesophageal reflux disease (GERD), however it should be appreciated that an end cap similar to any of the end caps discussed above can be used in the lower portion of the gastrointestinal tract for treating ailments such as fecal incontinence. The apparatus and method of the present invention preferably delivers radio frequency energy to the portion of such wall being treated. The apparatus and method provides consistent and repeatable treatments to the portion of such wall and utilizes a recess to shape the portion of such wall into a protrusion into which the energy is delivered. The apparatus and method of the invention utilizes vacuum to draw the portion of such wall into the recess in the apparatus and form a protrusion, and guides a stylet into the protrusion. A supply of an implant-forming material can optionally used with the apparatus and method for forming one or more implants in the wall being treated.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents.

What is claimed is:

1. An apparatus for treating a portion of a wall forming a cavity in a body having a natural body opening for accessing the cavity, comprising an elongate probe member having proximal and distal extremities, the distal extremity of the elongate probe member having an outer surface and being provided with at least one recess opening onto the outer surface and an internal passageway communicating with the recess, a suction source coupled to the proximal extremity of the elongate probe member and in communication with the passageway to create suction in the recess for drawing the portion of the wall into the recess, a stylet carried by the elongate probe member and extendable into the recess and an energy source coupled to the stylet for delivering energy through the stylet to the portion of the wall for treatment thereof.

2. The apparatus of claim 1 wherein the stylet is movably disposed in the elongate probe member and includes a distal end portion, the stylet being controllable from outside the body for movement from a retracted position in which the distal end portion of the stylet is out of the recess and an extended position in which the distal end portion of the stylet extends into the recess whereby the stylet can be extended into the portion of the wall drawn into the recess.

3. The apparatus of claim 2 wherein the elongate probe member has a lumen extending from the proximal extremity to the distal extremity, the stylet being slidably disposed in the lumen.

4. The apparatus of claim 1 wherein the stylet is fixed and nonmovably extends into the recess.

5. The apparatus of claim 1 wherein the elongate probe member has a lumen extending from the proximal extremity to the distal extremity permitting fluid communication between the suction source and the recess.

6. The apparatus of claim 1 comprising a plurality of stylers for penetrating the portion of the wall drawn into the recess, wherein the energy source is coupled to at least one stylet for delivering energy through the stylet to the portion of the wall for treatment thereof.

7. The apparatus of claim 1 wherein the stylet is a radio frequency electrode, further comprising a return electrode carried by the elongate probe member.

8. The apparatus of claim 7 wherein the radio frequency electrode is a needle electrode.

9. The apparatus of claim 8 wherein the return electrode is a needle electrode.

10. The apparatus of claim 1 wherein the energy source is a radio frequency generator.

11. The apparatus of claim 1 wherein the distal extremity has an outer cylindrical surface and the recess is an annular recess extending circumferentially around the outer cylindrical surface.

12. The apparatus of claim 11 comprising a plurality of stylers extending into the annular recess for penetrating the portion of the wall drawn into the recess, wherein the stylers are circumferentially spaced apart around the annular recess.

13. The apparatus of claim 12 wherein at least one stylet is an active electrode and at least one other stylet is a return electrode.

14. The apparatus of claim 1 wherein said stylet comprises an insulated surface and an energy delivery surface, wherein the energy source coupled to the stylet delivers energy through the energy delivery surface to the portion of the wall for treatment thereof.

15. An apparatus according to claim 1, wherein the elongate member includes an overtube member having proximal and distal extremities, the distal extremity including the outer surface, the overtube member having a length so that when the distal extremity is in the vicinity of a lower esophageal sphincter inside the body the proximal extremity is outside of the body.

16. An apparatus according to claim 1, wherein the elongate member includes an overtip assembly having proximal and distal extremities, the distal extremity including the outer surface, the overtip assembly having a length so that when the distal extremity is in the vicinity of the a lower esophageal sphincter inside the body the proximal extremity is inside the body.

17. An apparatus for use with a suction source and an energy source to treat a portion of a wall forming a cavity in a body having a natural body opening for accessing the cavity, comprising an elongate probe member having proximal and distal extremities, the distal extremity of the elongate probe member having an outer surface and being provided with at least one recess opening onto the outer surface and an internal passageway communicating with the recess whereby when the suction source is coupled to the apparatus a suction is created in the recess to draw the portion of the wall into the recess, a stylet extending into the recess for penetrating the portion of the wall drawn into the recess, the stylet being adapted for coupling to the energy source to deliver energy into the portion of the wall for treatment thereof.

18. The apparatus of claim 17 wherein the stylet is fixed and nonmovably extends into the recess.

19. The apparatus of claim 17 comprising a plurality of stylets for penetrating the portion of the wall drawn into the recess, wherein the energy source is coupled to at least one stylet for delivering energy through the stylet to the portion of the wall for treatment thereof.

20. The apparatus of claim 17 wherein the stylet is a radio frequency electrode, further comprising a return electrode carried by the elongate probe member and coupled to the energy source.

21. An apparatus for use with a suction source and an energy source to treat a portion of a wall forming a cavity in a body having a natural body opening for accessing the cavity, comprising an elongate probe member having proximal and distal extremities, the distal extremity of the elongate probe member having an outer surface and being provided with at least one recess opening onto the outer surface and an internal passageway communicating with the recess whereby when the suction source is coupled to the apparatus a suction is created in the recess to draw the portion of the wall into the recess, a stylet extendable into the recess for penetrating the portion of the wall drawn into the recess, a lead coupled to the stylet for permitting the stylet to be coupled to the energy source for delivering energy from the stylet into the portion of the wall for treatment thereof.

22. The apparatus of claim 21 wherein the stylet is movably disposed in the elongate probe member and includes a distal end portion, the stylet being controllable from outside the body for movement from a retracted position in which the distal end portion of the stylet is out of the recess and an extended position in which the distal end portion of the stylet extends into the recess whereby the stylet can be extended into the portion of the wall drawn into the recess.

23. An apparatus for treating a lower esophageal sphincter in a body having an esophagus, comprising a suction source, a radio frequency generator and an elongate probe member having proximal and distal extremities, the distal extremity of the elongate probe member having an outer surface and being provided with at least one recess opening onto the outer surface and an internal passageway communicating with the recess whereby when the suction source is activated a suction is created in the recess to draw a portion of the lower esophageal sphincter into the recess, the recess being formed at least in part by a base, a stylet extending upwardly from the base into the recess for penetrating the portion of the wall drawn into the recess, the stylet coupled to the energy source to deliver radio frequency energy into the lower esophageal sphincter when the radio frequency generator is activated for treatment of the lower esophageal sphincter.

24. The apparatus of claim 23 wherein the stylet is movably disposed in the elongate probe member and includes a distal end portion, the stylet being controllable from outside the body for movement from a retracted position in which the distal end portion of the stylet is out of the recess and an extended position in which the distal end portion of the stylet extends into the recess whereby the stylet can be extended into the portion of the lower esophageal sphincter drawn into the recess.

25. The apparatus of claim 23 wherein the stylet is fixed and nonmovably extends into the recess.

26. A method for treating a portion of a wall forming a cavity in a body having a natural body opening for accessing the cavity with an elongate probe member having a distal extremity provided with a recess therein and a stylet extendable into the recess, comprising the steps of supplying suction to the recess to draw the portion of the wall into the recess, extending the stylet into the portion of the wall drawn into the recess and delivering energy to the stylet to treat the wall.

27. The method of claim 26 wherein the delivering step includes the step of delivering radio frequency energy to the stylet.

28. The method of claim 26 wherein the extending step includes the step of moving the stylet from a retracted position in which the stylet is out of the recess to an extended position in which the stylet extends into the recess.

29. The method of claim 26 wherein the extending step occurs while the portion of the wall is being drawn into the recess.

30. The method of claim 26 further comprising the step of forming an implant in the portion of the wall drawn into the recess.

31. The method of claim 30 wherein the implant includes a medicament.

32. The method of claim 30 further comprising the step of injecting a conditioning solution into the portion of the wall drawn into the recess for facilitating the treatment.

33. The method of claim 26 further comprising the step of visualizing the cavity through the distal extremity of the elongate probe member.

34. The method of claim 26 wherein the supplying step includes the step of supplying suction to the recess to draw a portion of the wall forming the upper gastrointestinal tract in the vicinity of the lower esophageal sphincter into the recess and wherein the delivering step includes the step of delivering energy to the stylet to treat gastroesophageal reflux disease.

35. The method of claim 26 wherein the supplying step includes the step of supplying suction to the recess to draw a portion of the wall forming the lower gastrointestinal tract in the vicinity of the anal sphincter into the recess and wherein the delivering step includes the step of delivering energy to the stylet to treat fecal incontinence.
